(19) United States
(12) Patent Application Publication
(10) Pub. No.: US 2024/0400745 A1
(43) Pub. Date: Dec. 5, 2024

(54) METHOD FOR THE COUPLED PRODUCTION OF POLYURETHANES WITH REDUCED CO2 FOOTPRINT

(71) Applicant: Volkswagen Aktiengesellschaft, Wolfsburg (DE)

(72) Inventors: Armin ANIOL, Braunschweig (DE); Fabian FISCHER, Hannover (DE); Christian MAAS, Schwuelper (DE); Marc RUEGGEBERG, Braunschweig (DE)

(73) Assignee: Volkswagen Aktiengesellschaft, Wolfsburg (DE)

(21) Appl. No.: 18/805,502

(22) Filed: Aug. 14, 2024

Related U.S. Application Data
(63) Continuation of application No. PCT/EP2023/053644, filed on Feb. 14, 2023.

(30) Foreign Application Priority Data
Feb. 14, 2022 (DE) 10 2022 201 519.3

(51) Int. Cl. C08G 18/32

(52) U.S. Cl. CPC C08G 18/3203

(57) ABSTRACT

A method for the coupled production of polyurethanes. Polyurethane can be produced with a reduced CO₂ footprint via an energetic combination of the polyurethane synthesis with preceding process steps.

This nonprovisional application is a continuation of International Application No. PCT/EP2023/053644, which was filed on Feb. 14, 2023, and which claims priority to German Patent Application No. 10 2022 201 519.3, which was filed in Germany on Feb. 14, 2022, and which are both herein incorporated by reference.

### BACKGROUND OF THE INVENTION

### Field of the Invention

The invention relates to a method for the coupled production of polyurethanes with reduced CO₂ footprint for use in a motor vehicle.

### Description of the Background Art

The use of renewable raw materials is a promising leverage tool for improving the overall CO₂ balance of vehicles. In this context, sustainable polymer approaches are becoming increasingly important in the automotive industry.

Essentially the following strategies may be mentioned: polymers based on renewable raw materials, and polymers that are recovered as recycled plastics.

Polymers based on renewable raw materials, i.e., derived from biobased polymers, are known for keeping the CO₂ footprint low over the entire product life cycle, compared to the petrochemical alternative. According to the second alternative, polymers from recycling processes are used to keep the CO₂ footprint low via a closed materials cycle.

However, these two polymer classes are not suitable for use in applications with demanding requirements, since for the biobased approaches as well as the recycled plastics, due to natural synthesis processes on the one hand and degradation effects in the recycling process on the other hand, it is not possible to control the molecular weight, and thus the physical, mechanical, and chemical properties.

### SUMMARY OF THE INVENTION

It is therefore an object of the invention to provide a method for the production of polymers that are sustainable and have an advantageous property profile.

The object is achieved, in an example, by a method for the production of polyurethane, comprising: the recovery of atmospheric CO₂ in a first method step; the production of a diol compound (diol monomer) from the recovered atmospheric CO₂ in a second method step; and/or the polycondensation of the diol compound to form polyurethane in a third method step, wherein the third method step is energetically coupled with the second method step and/or the first method step.

The polymers based on atmospherically bound CO₂, obtained using the method according to the invention, advantageously have a defined molecular weight and a defined molecular weight distribution. A property profile that is specific for the particular application may thus be created which does not differ chemically from petrochemical plastic approaches, but which has a negative CO₂ balance compared to petrochemical polymer approaches.

Overall, by use of the method according to the invention, thermoplastic polymers based on bound CO₂ may thus be prepared which, in addition to the properties of ease of processability in forming processes, also have a property profile that is specific for the particular application and an improved CO₂ balance over the product life cycle.

US 2020/0291901 A1 and WO 2019/161114 A1 (which corresponds to US 2021/0120750), which are all herein incorporated by reference, describe options for recovering atmospheric CO₂. This CO₂ may be used for synthesis purposes.

The recovery of atmospheric CO₂ for synthesis purposes is energy-intensive. The recovery of a ton of CO₂ typically requires large quantities of electrical energy as well as thermal energy.

In addition, the second step, the production of a diol compound (in particular a polymerizable diol monomer) from the recovered atmospheric CO₂ proceeds in an endergonic manner and requires the supplying of energy.

The method according to the invention now advantageously provides for coupling the thermodynamic driving force of the polyurethane formation with the first and/or the second method step, i.e., the production of the diol compound, and/or particularly preferably coupling it with the recovery of atmospheric CO₂.

The energetic coupling of the steps may take place either directly by supplying the thermal energy that is generated in the third step, and/or by conversion into other forms of energy, in particular electrical energy.

In particular, the energetic coupling may take place directly by supplying the thermal energy that is generated in the third step, in the sense of thermal coupling of the steps via a combined process with heat exchange. This form of coupling is preferred, since the thermal energy may be utilized directly for recovering the CO₂ from the air.

Alternatively, a conversion into other forms of energy, in particular electrical energy, is possible. The conversion of thermal energy into electrical energy may take place via turbines or thermoelectrical generators, for example. The thermal energy that has been recovered in the third method step may initially be converted into electrical energy and subsequently used for the electrochemical synthesis of the diol and/or the isocyanate, or in particular also for the recovery of the CO₂.

The energetic coupling can take place via direct thermal coupling and also by conversion into electrical energy. The direct air capture (DAC) process, which requires both thermal energy and electrical energy, in the sense of an optimal combined process may thus be implemented in a very particularly preferred manner.

The feature of energetically coupling the third method step with the second method step and/or the first method step represents a particular advantage of the method according to the invention. Polymer production based on the CO₂ contained in the atmosphere may take place without eliminating these advantages due to the increased energy expenditure that occurs in the first method step for the recovery of the atmospheric CO₂, and/or in the second method step for the synthesis of the diol compound.

A method is described according to an example, in which the first method step includes the removal/extraction of CO₂ from ambient air.

A further method is described according to an example, in which the first method step is designed as a direct air capture process.

A further method is described according to an example, in which the diol compound according to the second method step includes at least one compound selected from 2,3-furandiol, propylene glycol, and/or monoethylene glycol, with 2,3-furandiol being preferred.

A further method is described according to an example, in which the production of the diol compound from the recovered atmospheric CO₂ can take place via electrochemical reduction.

Since the energetic coupling of the steps may take place by converting the thermal energy generated in the third step into electrical energy, an electrochemical reaction in the second step is advantageous, since this allows process-efficient energetic coupling of the processes.

A further method is described according to an example, in which the production of the diol compound from the recovered atmospheric CO₂ can be carried out using a catalyst, preferably a transition metal catalyst, particularly preferably a nickel catalyst.

The obtained polyurethane can be incorporated as a material in a motor vehicle.

The first method step, which relates to the recovery of CO₂ from the atmosphere, is described in greater detail below:

This step encompasses the extraction of carbon dioxide from the atmosphere. This process cycle can be divided into the substeps sorption and desorption.

For this purpose, initially the ambient air with the resulting CO₂ content is led through a filter unit, and the carbon dioxide is separated from the ambient air using suitable sorbents.

Thermal desorption subsequently takes place. The thermal desorption can be carried out at 70° C. to 180° C., and is used to separate the bound carbon dioxide from the sorbent.

The first method step described here for recovery of atmospheric CO₂ requires energy. Both process steps of the sorption cycle are considered from an energy standpoint. The sorption of a ton of CO₂ typically requires several hundred kWh of electrical energy, and often several times this amount of thermal energy, for the extraction.

In particular, the direct air capture (DAC) process is used in the described process. For this purpose, initially the ambient air, preferably containing 400 ppm CO₂, is led through a filter unit of the DAC facility, and the carbon dioxide is separated from the ambient air using suitable sorbent materials. Physisorption processes as well as chemisorption processes which separate the carbon dioxide from the ambient air may act here. Thermal desorption subsequently takes place, preferably at 100° C., for separating the bound carbon dioxide from the sorbent. Both process steps of the sorption cycle are considered from an energy standpoint.

The sorption of a ton of CO₂ typically requires 350 kWh of electrical energy and 1720 to 3350 kWh of thermal energy for the process steps. In particular, the electrical energy required for this purpose may preferably be obtained by energetic coupling with the third method step.

With regard to the thermal energy, the energetic coupling may take place directly by supplying the thermal energy that is generated in the third step, in the sense of thermal coupling of the steps via a combined process with heat exchange. This form of coupling is advantageous, since the thermal energy may be utilized directly for recovering the CO₂ from the air. The electrical energy may be provided via partial conversion of the thermal energy. The thermal energy that has been recovered in the third method step may initially be converted into electrical energy and subsequently used to provide the electrical energy that is required in the sorption process. In this regard, the method represents an optimal combined process.

The method is described according to an example in which polyethylenimine is used as sorbent material in the DAC process. It has been found that polyethylenimine has appropriate thermal conductivity properties for the described energetic coupling of the method steps.

The separated carbon dioxide can have a purity of >98%, and is used for the monomer synthesis in the second method step.

The second method step, which relates to the monomer synthesis, is described below.

The monomers contain two hydroxide groups. According to an example, the monomer contains at least one selected from 2,3-furandiol, propylene glycol, and/or monoethylene glycol.

The production of the starting materials takes place under mild reaction conditions and voltages ≤20 mV. 2,3-Furandiol, and likewise propylene glycol and monoethylene glycol, may be synthesized using an analogous catalytic process.

According to an example, a method for the production of polyurethane is described, wherein in a second method step a diol compound, for example 2,3-furandiol, is obtained by reduction of CO₂. The 2,3-furandiol is thus advantageously produced from a non-fossil carbon source.

According to an example, a method for the production of polyurethane is described in which the CO₂ is atmospheric CO₂. The described thermoplastic polymers therefore advantageously have a reduced or even negative CO₂ balance over the product life cycle. The described method thus contributes to improvement of the overall CO₂ balance of vehicles. The obtained polymers are sustainable polymers from the biosphere cycle, which are becoming increasingly important in the automotive industry. This is achieved by the polymer that is described according to this example.

According to an example, a method for the production of polyurethane is described, wherein the second method step (monomer synthesis) takes place electrochemically.

In particular, the electrical energy required for this purpose may preferably be obtained by energetic coupling with the third method step.

The electrochemical reduction of carbon dioxide, using water as a hydrogen source, may therefore enable sustainable production of the polymers from renewable energy sources.

According to an example, a method for the production of polyurethane is described, wherein the second method step is carried out using a catalyst. The overall reaction process may be advantageously speeded up significantly by means of transition metal catalysis. A heterogeneous catalytic operating principle of the transition metal is to be assumed.

According to an example, a method for the production of polyurethane is described, wherein the second method step further comprises:
(a) placing a catalyst-coated cathode together with an anode in an electrolyte;
(b) bringing the anode and the cathode into conductive contact with an external power source;
(c) providing a source of carbon dioxide for the cathode; and
(d) using the external source of electricity to drive an electrolysis reaction at the cathode, in which hydrocarbons, products, or both are produced from the carbon dioxide. Reference is made to U.S. Pat. No. 10,676,833 B2, which is incorporated herein by reference.

The third method step, which relates to the polymer synthesis, is described below.

Since the synthesized monomer products contain two hydroxide groups, these may be utilized in the third method step for the synthesis of polyurethanes.

The selection of the appropriate monomer structure is significant in determining the material properties of the polyurethane. Monoethylene glycol results in a soft polymer having a low glass transition temperature, whereas 2,3-furandiol produces a hard, brittle material having a high glass transition temperature.

With regard to the property profiles described above, the propylene glycol is between monoethylene glycol and 2,3-furandiol.

For this purpose, the diol structures are crosslinked via a polycondensation reaction to form the required polyols, and are used as a starting material for the polymer synthesis. In the following discussion, the monomers are referred to as polyols, regardless of the chemical structure, on account of the described end groups. By use of the previously synthesized polyols and diisocyanates, the polyurethane structures and the polymeric network are subsequently built via polyaddition reactions and the formation of a urethane group. The synthesis of the polyurethane formation proceeds in a strongly exothermic reaction (temperature generally greatly above 100° C.). This heat of reaction is utilized in the described process chain for the sorption, desorption, and monomer synthesis.

The method is described according to an example, in which the production of an isocyanate compound takes place in a further method step with reduction of the CO₂ recovered in the first method step. The method is described according to an example, in which the reduction of the atmospherically recovered CO₂ takes place by transition metal catalysis or electrochemically.

The method is described according to an example, in which the third method step is energetically coupled with the further method step, which includes the production of the isocyanate compound.

The method is described according to an example, in which the isocyanate compound in the third method step is used to prepare the polyurethane.

Overall, the invention thus describes a method for polyurethane production, wherein the CO₂ is atmospheric CO₂. Whether or not the polyurethane has been obtained using atmospheric CO₂ may be characterized analytically. In particular in isotope measurement, the ratio of two different types of carbon atoms that may occur in CO₂ molecules is determined: ¹³C and ¹²C, where the number characterizes the mass of the atom. Fossil fuels, for example, are characterized by a low ¹³C to ¹²C ratio. In contrast, atmospheric CO₂ has a higher ¹³C to ¹²C ratio. The ¹³C and ¹²C isotope distribution thus represents a type of “fingerprint” for forming the product from atmospheric CO₂.

According to a further aspect of the invention, the use of polyurethane in a motor vehicle is described.

The various examples of the invention set forth in this patent application, unless indicated otherwise in the individual case, are advantageously combinable with one another.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1a shows a second method step for forming diol compounds, in particular 2,3-furandiol, by electrochemical reduction of CO₂,

FIG. 1b shows a subordinate second method step for forming isocyanate from CO₂, and

FIG. 2 shows a third method step for forming polyurethanes by a polymerization reaction of the diol compounds, to be condensed to form polyols, with diisocyanates.

### DETAILED DESCRIPTION

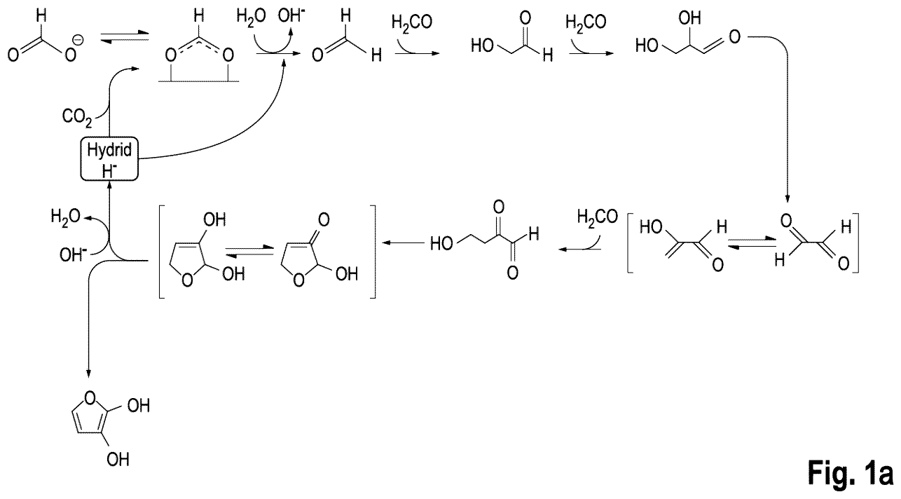

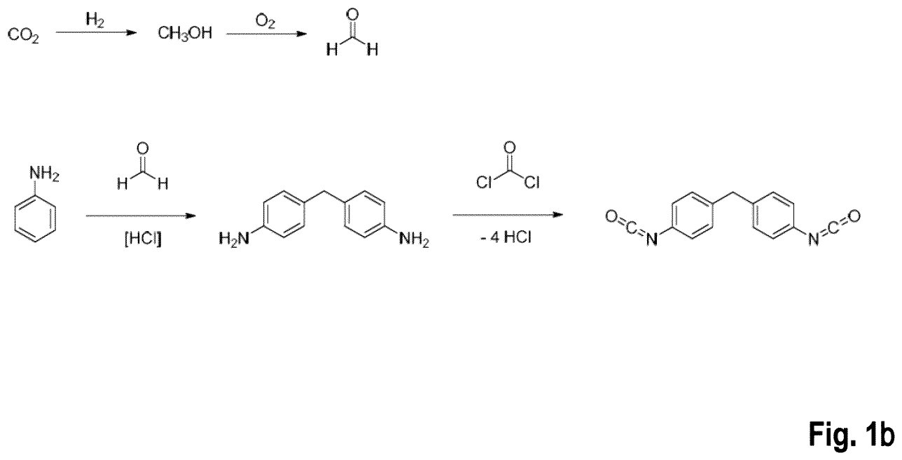

FIG. 1a shows how, after separation of the CO₂ in the first method step, a diol compound, in the present case 2,3-furandiol, is obtained in a second method step by electrochemical reduction of the atmospheric CO₂.

From a mechanistic standpoint, it is assumed that the CO₂ reduction takes place by catalysis via the illustrated mechanism. In substep 1, CO₂ is inserted into a surface hybrid bond to produce an adsorbed formate species. It is assumed that this is the potential-determining step (PDS).

In substep 2, the absorbed formate is protonated and attacked by a second hydride. Formaldehyde is formed after hydroxide splits off. Formaldehyde is not detected, since the formaldehyde that forms is highly reactive.

Two successive thermodynamically preferred aldehyde condensation reactions are then postulated for the production of glyceraldehyde. The keto-enol tautomerization presumably has the highest energy barrier, and thus explains the accumulation of the methylglyoxal precursor. This step is followed by a further condensation of aldehyde with formaldehyde on the catalyst. The cyclization forms the stable five-membered ring by intramolecular condensation of an alcohol and an aldehyde. The hydride abstraction, the reaction to form the end product, is driven by the stability of the aromatic furan ring.

FIG. 1b shows a subordinate method step for forming isocyanate from CO₂. This step is referred to here as a “subordinate second method step” because this step, the same as the reaction step shown in FIG. 1a, takes place after the first method step, i.e., the recovery of CO₂ from the atmosphere, but before the third method step, i.e., the polyurethane formation. This step may take place concurrently with, before, or after the diol synthesis step.

The same as the reaction step for diol production shown in FIG. 1a, the CO₂ recovered from the atmosphere is also used for the isocyanate synthesis in FIG. 1b.

The production of formaldehyde from atmospheric CO₂ is initiated by a reduction of atmospheric carbon dioxide, using hydrogen, to give a carbon monoxide intermediate which forms the methanol via further reduction steps with hydrogen. This reaction may take place via transition metal catalysis, or by direct electrochemical means using suitable electrocatalysts, without forming a carbon dioxide intermediate. Suitable catalysts include Ni, Fe, Ag, and Cu-based phosphorus compounds. Subsequent oxidation of the compound results in formaldehyde.

The production of the isocyanate initially takes place via the reaction of aniline with the CO₂-based formaldehyde in acidic medium to give diaminophenylmethane, which is subsequently reacted with phosgene to give diphenylmethane-4,4′-diisocyanate.

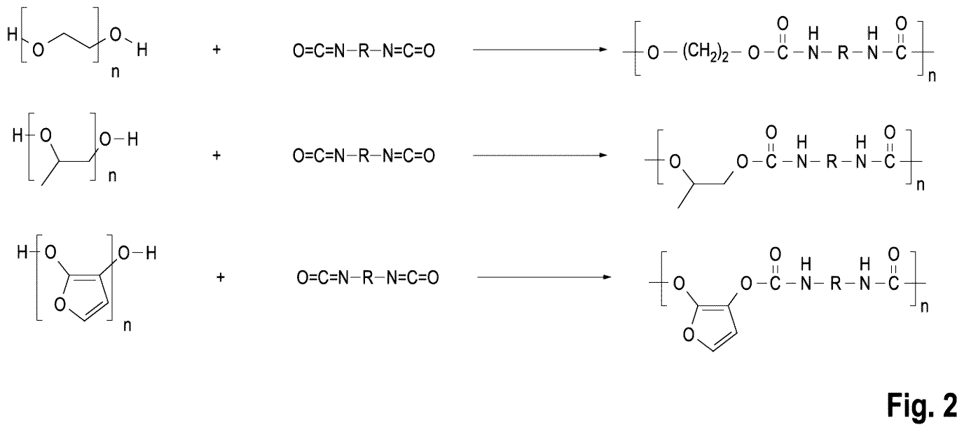

FIG. 2 shows the subsequent polyurethane formation. In this third method step for forming polyurethanes, a polymerization reaction of the diol compounds, created in the second step, with diisocyanates is carried out. Corresponding polyols are initially formed, which then react with the diisocyanates. In addition to 2,3-furandiol, as shown in FIG. 1a, propylene glycol and monoethylene glycol may likewise be synthesized using an analogous catalytic process. The synthesized monomer products all contain two hydroxide groups, which in the process step shown in FIG. 2 may be used for the synthesis of polyurethanes. The selection of the appropriate monomer structure is significant in determining the material properties of the polyurethane. Monoethylene glycol results in a soft polymer having a low glass transition temperature, whereas 2,3-furandiol produces a hard, brittle material having a high glass transition temperature. In the property profiles described above, the propylene glycol is between monoethylene glycol and 2,3-furandiol.

For this purpose, the diol structures are crosslinked via a polycondensation reaction to form the required polyols, and are used as a starting material for the polymer synthesis.

The polymerization of the polyols and isocyanates takes place as a polycondensation under relatively mild reaction conditions. By use of the previously synthesized polyols and diisocyanates, the polyurethane structures and the polymeric network are subsequently built via polyaddition reactions and the formation of a urethane group. The polyurethane formation proceeds in a stepwise manner, wherein a bifunctional molecule having an isocyanate group and a hydroxide group is initially formed from a diol/polyol and a diisocyanate. In further synthesis steps, oligomeric structures are built from the bifunctional molecule, using further monomers. In a manner analogous to the conventional polyurethane systems, crosslinking of the polymer chains takes place, for example forming allophanate structures from a reaction of an isocyanate with a urethane group, via multiple use of amines and trimerization reactions of isocyanate structures to give isocyanurates. The synthesis of the polyurethane formation proceeds in a strongly exothermic reaction at a temperature that is generally greatly above 100° C. This heat of reaction is utilized in the described process chain for the sorption, desorption, and monomer synthesis from the first method step and/or the second method step in the sense of energetic coupling.

Whether or not the polyurethane has been obtained using atmospheric CO₂ according to method steps 1 and 2 may be characterized analytically by isotope measurement. In isotope measurement, the ratio of two different types of carbon atoms that may occur in CO₂ molecules is determined: ¹³C and ¹²C, where the index number characterizes the mass of the atom. Atmospheric CO₂ has a higher ¹³C to ¹²C ratio, so that the ¹³C and ¹²C isotope distribution represents a type of “fingerprint” for forming the product from atmospheric CO₂.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A method for the production of polyurethane, the method comprising:
recovering atmospheric CO₂ in a first method step;
producing a diol compound from the recovered atmospheric CO₂ in a second method step; and
polycondensating the diol compound to form polyurethane in a third method step,
wherein the third method step is energetically coupled with the second method step and/or the first method step.

2. The method according to claim 1, wherein the first method step includes the sorption and desorption of the atmospheric CO₂.

3. The method according to claim 1, wherein the first method step is designed as a direct air capture process.

4. The method according to claim 1, wherein polyethylenimine is used as sorbent material.

5. The method according to claim 1, wherein the diol compound according to the second method step includes at least one compound selected from 2,3-furandiol, propylene glycol, and/or monoethylene glycol.

6. The method according to claim 1, wherein the production of the diol compound from the recovered atmospheric CO₂ takes place via electrochemical reduction.

7. The method according to claim 1, wherein in a further method step the production of an isocyanate compound takes place with reduction of the CO₂ recovered in the first method step.

8. The method according to claim 1, wherein the third method step is energetically coupled with the further method step, which includes the production of the isocyanate compound.

9. The method according to claim 7, wherein the isocyanate compound in the third method step is used to prepare the polyurethane.

10. The method according to claim 7, wherein the reduction of the atmospherically recovered CO₂ takes place by transition metal catalysis.

11. The method according to claim 7, wherein the reduction of the atmospherically recovered CO₂ takes place electrochemically.

12. The method according to claim 1, wherein the obtained polyurethane is incorporated as a material in a motor vehicle.
